(19) United States
(12) Patent Application Publication
(10) Pub. No.: US 2019/0159823 A1
(43) Pub. Date: May 30, 2019

(54) PRE-SURGICAL PLANNING APPARATUS AND PRE-SURGICAL PLANNING METHOD

(71) Applicant: Metal Industries Research & Development Centre, Kaohsiung (TW)

(72) Inventors: Szu-Hua Yang, Kaohsiung City (TW); Chien-Chang Chen, Kaohsiung City (TW); Yii-Der Wu, Kaohsiung City (TW)

(73) Assignee: Metal Industries Research & Development Centre, Kaohsiung (TW)

(21) Appl. No.: 15/823,597

(22) Filed: Nov. 28, 2017

(51) Int. Cl. A61B 18/10; A61B 18/08; G06F 19/00

(52) U.S. Cl. CPC A61B 18/10; A61B 2018/00988; G06F 19/3437; A61B 18/082

(57) ABSTRACT

A pre-surgical planning apparatus adapted to estimate ablation information of a target object ablated by a magnetic heat treatment device is provided. The magnetic heat treatment device includes a needle and a coil. The pre-surgical planning apparatus includes a storage device, a processing device, and an input device. The storage device stores an estimation model. The input device provides a plurality of parameters including a needle angle parameter, a needle characteristic parameter, and a then gal conductivity of the target object. The processing device inputs the parameters to the estimation model, such that the processing device executes the estimation model to estimate the ablation information of the target object ablated by the magnetic heat treatment device with a preset power and a preset length of time. The ablation information includes temperature curve information and temperature distribution information. Besides, a pre-surgical planning method is also provided.

### BACKGROUND OF THE INVENTION

### 1. Field of the Invention

The invention relates to a planning technology. More particularly, the invention relates to a pre-surgical planning apparatus and a pre-surgical planning method.

### 2. Description of Related Art

Cancer (also known as tumor) is one of major human diseases ranked as the top three of statistical death factors in many countries. Thus, not only is cancer treatment an urgent medical need in those countries, research and development on various pieces of medical equipment for cancer-related treatment has also becomes very important in the related field. In particular, a thermotherapy surgery on tumor is currently one of main cancer treatment techniques. For example, the main cancer treatment techniques, such as RFA (Radio Frequency Ablation) or MWA (Microwave Ablation) in tumor ablation surgery, are now applicable in local tumor treatment.

On the other hand, a thermotherapy system based on EMA (Electromagnetic Ablation) is also available. However, because the current magnetic heat treatment system is still lack of an estimation technique for post-ablation temperature area range, medical professionals are unable to clearly learn about ablation conditions at the site of treatment for patients so proper commands or operations cannot be promptly given to the thermotherapy system. For instance, said ablation conditions can involve an amount of current to go through a magnetic field generator, a length of an ablation time, a needle tip depth and angle for inserting a needle, whether an ablation range on a biological tissue to be ablated meets the criteria, and the like. In other words, if physical characteristics of the biological tissue an operation time on which cannot be precisely handled in the practice, a normal tissue may be inadvertently removed since a diameter of high temperature area generated by energy-based surgical instruments may become overly large. Alternatively, a complete ablation result cannot be effectively achieved if the diameter of the high temperature area is overly small.

Accordingly, it is required to ensure that the electromagnetic ablation can provide a safe treatment range in order to improve a treatment quality as well as surgical safety and accuracy for patients. Therefore, finding a way to effectively estimate a temperature area range and a temperature variation of the needle over time during the electromagnetic ablation is one of important issues to be addressed. In view of the above, several embodiments of the invention are provided as follows.

### SUMMARY OF THE INVENTION

The invention provides a pre-surgical planning apparatus and a pre-surgical planning method capable of effectively estimating ablation information of a target object ablated by a magnetic heat treatment device.

In an embodiment of the invention, a pre-surgical planning apparatus is adapted to estimate ablation information of a target object ablated by a magnetic heat treatment device. The magnetic heat treatment device includes a needle and a coil. The pre-surgical planning apparatus includes a storage device, a processing device, and an input device. The storage device is configured to store an estimation model. The processing device is coupled to the storage device. The processing device is configured to read the estimation model. The input device is coupled to the processing device. The input device is configured to provide a plurality of parameters. The parameters include a needle angle parameter, a needle characteristic parameter, and a thermal conductivity of the target object. The processing device inputs the parameters to the estimation model, such that the processing device executes the estimation model to estimate the ablation information of the target object ablated by the magnetic heat treatment device with a preset power and a preset length of time. The ablation information includes temperature curve information and temperature distribution information.

In an embodiment of the invention, the pre-surgical planning method is adapted to estimate ablation information of a target object ablated by a magnetic heat treatment device. The magnetic heat treatment device includes a needle and a coil. The pre-surgical planning method includes obtaining a plurality of parameters through an input device, wherein the parameters include a needle angle parameter, a needle characteristic parameter, and a thermal conductivity of the target object; and inputting the parameters to an estimation model, and executing the estimation model to estimate the ablation information of the target object ablated by the magnetic heat treatment device with a preset power and a preset length of time, wherein the ablation information includes temperature curve information and temperature distribution information.

To sum up, in the pre-surgical planning apparatus and the pre-surgical planning method provided by the embodiments of the invention, the corresponding estimation result may be generated through the estimation model according to the parameters provided by the input device. The estimation model is the deep neural network. The pre-surgical planning apparatus provided by the embodiments of the invention trains the estimation model in advance through a nonlinear regression, such that the estimation model can provide accurate ablation information. The ablation information includes the temperature curve information and the temperature distribution information.

To make the aforementioned and other features and advantages of the invention more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 illustrates a schematic view of a pre-surgical planning apparatus in an embodiment of the invention.

FIG. 2 illustrates a schematic view of an estimation model in an embodiment of the invention.

FIG. 3 illustrates a schematic view of a needle in an embodiment of the invention.

FIG. 4 illustrates a schematic view of a magnetic heat treatment device in an embodiment of the invention.

FIG. 5 illustrates a flowchart of a pre-surgical planning method in an embodiment of the invention.

FIG. 6A illustrates a schematic diagram of temperature curve information in an embodiment of the invention.

FIG. 6B illustrates a schematic diagram of temperature distribution information in an embodiment of the invention.

FIG. 7 illustrates a flowchart of a pre-surgical planning method in another embodiment of the invention.

### DESCRIPTION OF THE EMBODIMENTS

In order to make the invention more comprehensible, several embodiments of the invention are introduced herein to describe the invention, but the invention is not limited by the embodiments. Suitable combinations among the embodiments are also allowed. Moreover, elements/components/steps with the same reference numerals are used to represent the same or similar parts in the drawings and embodiments.

FIG. 1 illustrates a schematic view of a pre-surgical planning apparatus in an embodiment of the invention. Referring to FIG. 1, a pre-surgical planning apparatus 100 includes a processing device 110, a storage device 120, and an input device 130. In the present embodiment, the pre-surgical planning apparatus 100 estimates ablation information of a target object ablated by a magnetic heat treatment device. Further, the magnetic heat treatment device includes a needle and a coil. In the present embodiment, the storage device 120 stores an estimation model 121. The input device 130 is configured to provide a plurality of parameters. Further, the parameters include a needle angle parameter, a needle characteristic parameter, and a thermal conductivity of the target object. Nevertheless, in an embodiment, the parameters may also be obtained through automatic detection of a needle positioning system and thus are not limited to be provided by the input device 130 only.

In the present embodiment, a user may set a target temperature to the estimation model 121 through the input device 130, and the processing device 110 inputs the parameters to the estimation model 121. The processing device 110 executes the estimation model 121, such that the estimation model 121 simulates a result of an electromagnetic heat ablation surgery. The processing device 110 executes the estimation model 121 to estimate the ablation information of the target object ablated by the magnetic heat treatment device with a preset power (e.g., 10 W of power recommended) and a preset length of time (e.g., 60 seconds of ablation time recommended) according to the target temperature. That is, the estimation model 121 simulates the needle induced by the coil, wherein the coil induces the needle according to the preset power, so that the coil heats the needle with a non-contact type method. In the present embodiment, the ablation information includes temperature curve information and temperature distribution information.

In the present embodiment, the target object is a biological tissue, and the biological tissue may be, for example, a vitro tissue or a living tissue. The biological tissue may be, for example, tissue parts of various organs in human or animal body, such as the thyroid or liver tissue, which are not particularly limited by the invention. In the present embodiment, the biological tissue may include the fat tissue, and a thermal transfer effect of the biological tissue is to be more evident as affected by the fat tissue. As such, the thermal conductivity of the target object provided by the present embodiment is determined by a body fat parameter of the target object. To be specific, a thermal conductivity equation of the biological tissue may be presented by Equation (1) below. In the present embodiment, the estimation model 121 may perform relative estimation operations by applying, for example, the Equation (1) below, wherein a symbol Q is a heat energy, a symbol A is a sectional area of the biological tissue, a symbol d is a thickness of the biological tissue, a symbol (T2−T1) is a temperature difference before and after being heated, and a symbol k is the thermal conductivity. For instance, if the target object is a specific tissue part in human body, and the thermal conductivity of fat of the specific tissue part is approximately 20% of that of water, k is thus approximately equal to 0.12 Wm⁻¹ k⁻¹.

$$\frac{dQ}{dt} = k \times A \times ( T 2 - T 1 ) / d \qquad Equation ( 1 )$$

In the present embodiment, medical professionals may further provide a location parameter and parameters of a target temperature and a heating rate of the coil in the target object of a magnetic portion of the needle through the input device 130, such that more influential parameters may be taken into consideration by the estimation model 121 and estimation may thereby be accurately performed. In the present embodiment, the location parameter may include, for example, a needle tip depth, a needle tip distance, a coil radial distance, and other similar location parameters. In the present embodiment, the heating rate is, for example, 5 seconds to 90 seconds. The target temperature is, for example, 0 degree (° C.) to 120 degrees (° C.). Nevertheless, in an embodiment, medical professionals may further provide other related information not limited to the above, such as a model number of the needle, an expected treatment range, a respiratory rate, vibration, gender, or race. Moreover, in the present embodiment, the estimation model 121 may further recommends a power (e.g., 10 W) for driving the coil, such that medical professionals may accurately control the magnetic heat treatment device to perform the electromagnetic heat ablation surgery according to the recommended power.

In the present embodiment, the processing device 110 may be, for example, a central processing unit (CPU) composed of single-core or multi-core, a programmable microprocessor for general purpose or special purpose, a digital signal processor (DSP), a programmable controller, an application specific integrated circuits (ASIC), a programmable logic device (PLD) or other similar devices, or a combination of the above devices.

In the present embodiment, the storage device 120 may be, for example, a random access memory (RAM), a read-only memory (ROM), or a flash memory and the like, which may be used to store the data, the parameters, and the estimation model 121 described in each embodiment of the invention. A pre-surgical planning method described in each embodiment of the invention can be realized by the processing device 110 through reading the data, the parameters, and the estimation model 121 stored in the storage device 120.

In the present embodiment, the input device 130 may be, for example, a physical component, such as a physical keyboard, mouse, button or touchpad, and the like. Alternatively, the input device 130 may also be, for example, a software component such as an input interface. Moreover, the pre-surgical planning apparatus 100 may further include a display device in an embodiment. The display device may be, for example, a display has touch functions. Moreover, the display device can display image information of the input interface, such that medical professionals can input setting parameters by touching on the display device. Alternatively, medical professionals may also input the setting parameters by using an additional physical keyboard, but the invention is not limited to the above.

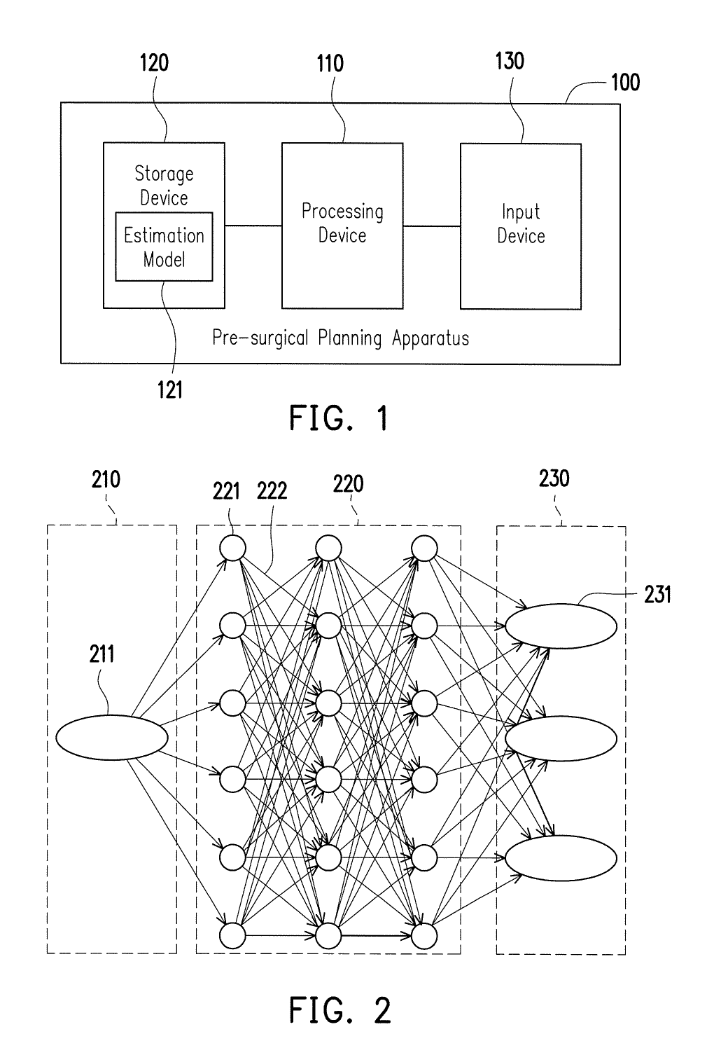

FIG. 2 illustrates a schematic view of an estimation model in an embodiment of the invention. With reference to FIG. 1 and FIG. 2, the processing device 110 trains or adjusts the estimation model 121 in advance through a nonlinear regression according to a plurality of pieces of sampling data. In the present embodiment, the estimation model 121 may be a neural network, for example, a deep neural network (DNN), a fuzzy neural network (FNN), and the like. The estimation model 121 may be, for example, a deep neural network as shown in FIG. 2 in this embodiment. The deep neural network of the estimation model 121 includes an input layer 210, a plurality of hidden layers 220, and an output layer 230. The hidden layers 220 of the estimation model 121 include a plurality of influential parameters 221 and a plurality of weights 222. To be specific, plural parameters provided by the input device 130 are used as input variables 211 of the input layer by the processing device 110, so as to perform calculation through the influential parameters 221 and the weights 222 of the hidden layers 220 and generate a plurality of output variables 231. Note that in the present embodiment, a number of the hidden layers most adapted to estimate the ablation information described in each embodiment of the invention is 3 to 60, which should however not be construed as limitations to the invention.

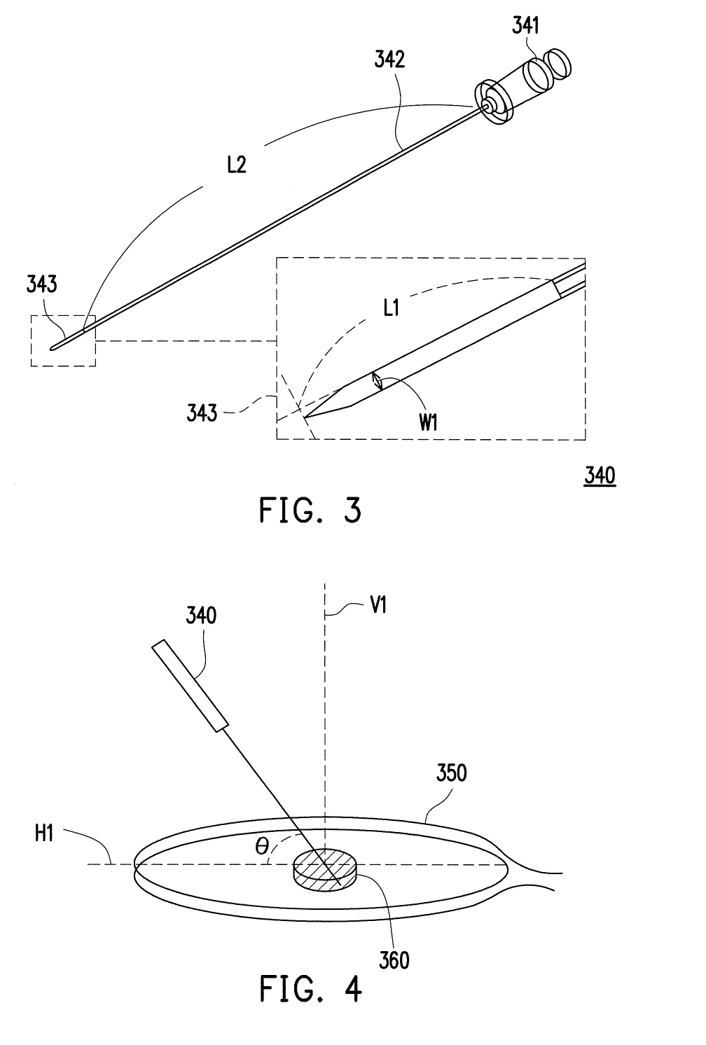

FIG. 3 illustrates a schematic view of a needle in an embodiment of the invention. Referring to FIG. 3, a needle 340 includes a holding portion 341, a non-magnetic portion 342, and a magnetic portion 343. The magnetic portion 343 may be made of a metal material with favorable magnetic properties. The magnetic portion 343 may be, for example, a solid medical grade metal made of materials like silver, platinum, stainless steel, titanium or titanium alloy, and the non-magnetic portion 342 may be, for example, made of materials like a medical grade ceramic. Moreover, a thermocouple element may further be included between the non-magnetic portion 342 and the magnetic portion 343 in an embodiment. The thermocouple element may be used to sense a temperature value of the magnetic portion 343.

To be specific, when the needle 340 is used in the electromagnetic heat ablation surgery, the magnetic portion 343 is placed in the target object by medical professionals and heat energy is generated according to a magnetic field provided by an external coil to ablate the target object. That is, a magnetic portion length L1 of the magnetic portion 343 and a needle diameter W1 may be determined according to different target objects to be ablated in this embodiment. Therefore, the needle characteristic parameter in each of the embodiments may include, for example, the magnetic portion length L1 and the needle diameter W1 of FIG. 3. Nevertheless, specifications of the needle 340 may vary according to different ablation treatments required in an embodiment. For instance, the magnetic portion length L1 is, for example, 5 mm, 7 mm, 10 mm, or 15 mm, and the needle diameter W1 of the needle 340 is, for example, 0.7 mm. Besides, a non-magnetic portion length L2 of the non-magnetic portion 342 is, for example, 70 mm to 90 mm, which are not particularly limited by the invention. The needle characteristic parameter is not related to the non-magnetic portion length L2 in this embodiment.

FIG. 4 illustrates a schematic view of a magnetic heat treatment device in an embodiment of the invention. The followings refer to FIG. 3 and FIG. 4. The magnetic heat treatment device includes the needle 340 and a coil 350. In the present embodiment, the coil 350 may be a magnetic focusing coil composed of a single-turn or a multi-turn metallic conductor and is configured to generate a magnetic field after being conducted. For instance, the coil 350 may receive an alternating current to generate an alternating magnetic field. The coil 350 induces the magnetic portion 343 of the needle 340 through the alternating magnetic field to enable the magnetic portion 343 of the needle 340 to generate heat energy. That is, when the needle 340 is used by medical professionals to perform the electromagnetic heat ablation surgery, the magnetic portion 343 is placed in the target object 360 and the magnetic portion 343 is surrounded by the coil 350 by the medical professionals, such that the magnetic portion 343 is located in a coil range of the coil 350. As such, the needle 340 may ablate the target object 360 through the heat energy generated by the magnetic portion 343.

The coil 350 is formed on a plane in this embodiment. A normal vector of the plane is parallel to a vertical axis V1, and a horizontal axis H1 is located on the plane. An angle θ is included between the needle 340 and the horizontal axis H1. In the present embodiment, the angle θ included between the needle 340 and the coil 350 may range between 0 degree and 90 degrees. That is, a magnetic induction effect provided by the coil 350 to the magnetic portion 343 of the needle 340 may be determined by the angle θ. As such, the needle angle parameter of each of the embodiments of the invention may refer to, for example, the angle θ of FIG. 4.

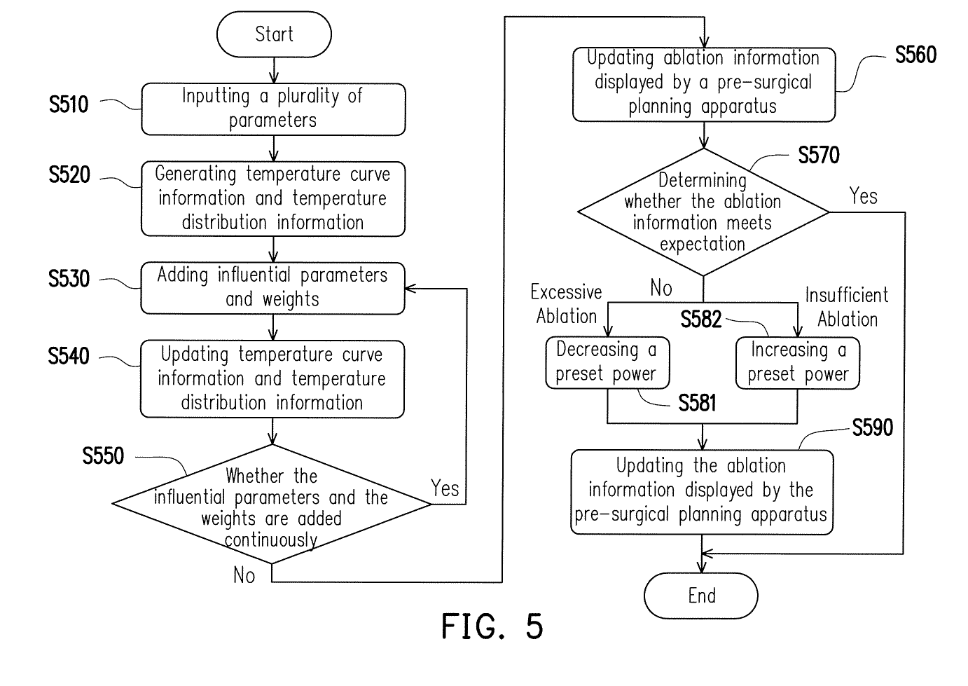

FIG. 5 illustrates a flowchart of a pre-surgical planning method in an embodiment of the invention. Referring to FIG. 1 and FIG. 5, the pre-surgical planning method of this embodiment may be adapted to the pre-surgical planning apparatus 100 of the embodiment of FIG. 1. In step S510, a user may input a plurality of parameters through the input device 130 of the pre-surgical planning apparatus 100. The parameters include a needle angle parameter, a needle characteristic parameter, and a thermal conductivity of the target object. In an embodiment, the user may further set a target temperature to the estimation model 121 through the input device 130, and that the estimation model 121 may perform estimation based on the target temperature. In step S520, the processing device 110 of the pre-surgical planning apparatus 100 inputs the parameters to the estimation model 121, such that the processing device 110 executes the estimation model 121 to generate the temperature curve information and the temperature distribution information. In step S530, the user may add influential parameters and weights through the input device 130. The influential parameters and the weights may be related to updates of, for example, the needle angle parameter, the needle characteristic parameter, or the thermal conductivity of the target object or may be related to, for example, adjustments of the estimation model 121. In step S540, the processing device 110 updates the temperature curve information and the temperature distribution information according to the influential parameters and the weights added. In step S550, the user may input a confirmation signal through the input device 130, such that, the processing device 110 may determine whether the user keeps adding influential parameters and weights according to the confirmation signal. If Yes is determined, the processing device 110 repeats step S530. If No is determined, the processing device 110 performs step S560.

In step S560, the processing device 110 updates the ablation information displayed by the pre-surgical planning apparatus 100 through the display device. In step S570, the user may input the confirmation signal through the input device 130, such that, the processing device 110 may determine whether the ablation information meets expectation according to the confirmation signal. If the confirmation signal inputted by the user is excessive ablation, the processing device 110 performs step S581 to decrease the preset power. If the confirmation signal inputted by the user is insufficient ablation, the processing device 110 performs step S582 to increase the preset power. Further, the processing device 110 performs step S590 so as to update the ablation information displayed by the pre-surgical planning apparatus through the display device according to the preset power adjusted and ends the pre-surgical planning. Nevertheless, in step S570, if the confirmation signal inputted by the user indicates that the ablation information meets expectation, the processing device 110 ends the pre-surgical planning.

Therefore, in the pre-surgical planning method of this embodiment, an ablation range may be estimated according to the parameters inputted by medical professionals, and a visualized estimation result is further provided. Medical professionals can thereby conveniently adjust a magnitude of the preset power driving the magnetic heat treatment device according to the visualized estimation result, so as to further adjust the ablation range effectively. In addition, in this embodiment, people having ordinary skill in the art may acquire sufficient teachings, suggestions, and other details related to the details of the device characteristics and details of the technology of the pre-surgical planning apparatus 100 according to content of the embodiments of FIG. 1 to FIG. 4, and that detailed descriptions are not further provided hereinafter.

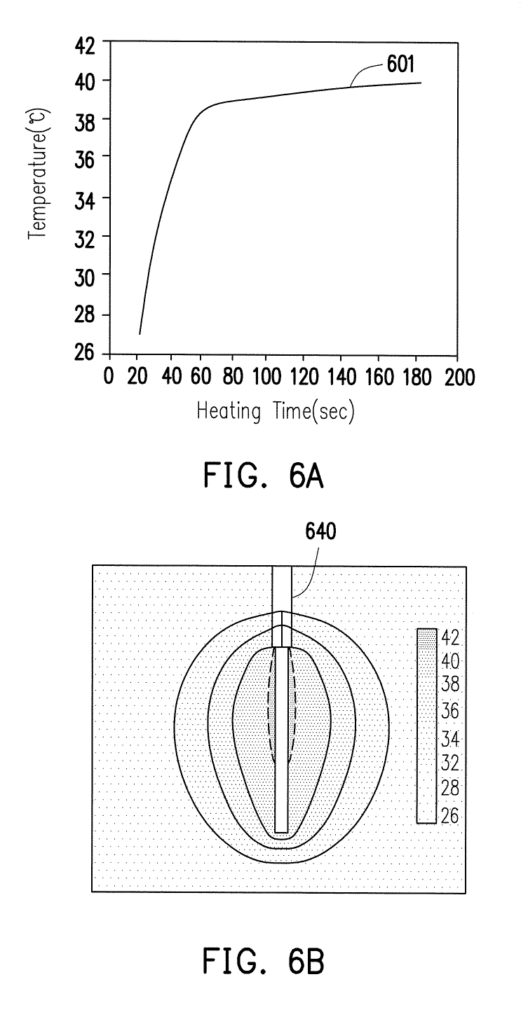

FIG. 6A illustrates a schematic diagram of temperature curve information in an embodiment of the invention. With reference to FIG. 1 and FIG. 6A, in the present embodiment, ablation estimation information of the target object ablated by the magnetic heat treatment device with the preset power and the preset length of time estimated by the processing device 110 through the estimation model 121 may include temperature curve information as shown in FIG. 6A. In the present embodiment, the pre-surgical planning apparatus 100 may display a human-computer interaction through the display device, and the human-computer interaction may include the temperature curve information of FIG. 6A. In the present embodiment, the temperature curve information includes a temperature variation curve 601 of the magnetic portion of the needle. Accordingly, medical professionals can effectively take a length of the ablation time into consideration according to the estimated temperature variation curve 601. Moreover, medical professionals can also correspondingly adjust the current magnitude for driving an electromagnetic coil, the length of the ablation time and the needle tip depth and angle of the needle for inserting the target object according to the temperature variation curve 601, so as to effectively control the ablation result of the target object. Incidentally, said ablation time refers to a time length between the beginning of driving the coil and the end of driving the coil.

FIG. 6B illustrates a schematic diagram of temperature distribution information in an embodiment of the invention. With reference to FIG. 1 and FIG. 6B, in the present embodiment, the ablation estimation information of the target object ablated by the magnetic heat treatment device with the preset power and the preset length of time estimated by the processing device 110 through the estimation model 121 may include the temperature distribution information as shown in FIG. 6B. In the present embodiment, the pre-surgical planning apparatus 100 may display the human-computer interaction through the display device, and the human-computer interaction may include the temperature curve information of FIG. 6B. In the present embodiment, the temperature distribution information may be a temperature envelope distribution diagram. Further, the temperature envelope distribution diagram may present temperature variations of a needle 640 generated in the target object (e.g., a temperature variation of 26° C. to 42° C. as shown in FIG. 6B). In this way, before the electromagnetic heat ablation surgery begins, medical professionals may learn about the possible ablation results and a size of the ablation range according to the temperature envelope distribution diagram presented in FIG. 6B as estimated by the pre-surgical planning apparatus 100, so as to effectively handle ablation-related settings.

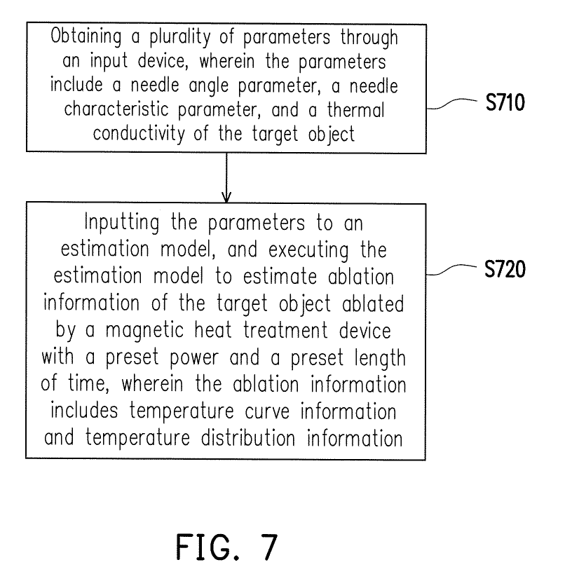

FIG. 7 illustrates a flowchart of a pre-surgical planning method in another embodiment of the invention. Referring to FIG. 1 and FIG. 7, the pre-surgical planning method of this embodiment may be adapted to the pre-surgical planning apparatus 100 of the embodiment of FIG. 1. In step S710, the processing device 110 of the pre-surgical planning apparatus 100 obtains a plurality of parameters through the input device 130, wherein the parameters include a needle angle parameter, a needle characteristic parameter, and a thermal conductivity of the target object. In step S720, the processing device 110 inputs the parameters to the estimation model 121, and executes the estimation model 121 to estimate the ablation information of the target object ablated by the magnetic heat treatment device with the preset power and the preset length of time. Further, the ablation information includes the temperature curve information and the temperature distribution information. Therefore, in the pre-surgical planning method of this embodiment, the possible ablation results generated by the magnetic heat treatment device in an actual treatment process may be effectively estimated. The user thus may adjust the setting parameters of the magnetic heat treatment apparatus in advance according to a planning result of the pre-surgical planning apparatus 100. In the present embodiment, the setting parameters of the magnetic heat treatment apparatus include, for example, a recommended power, a treatment range, a temperature curve, a needle angle parameter, a needle characteristic parameter, a recommended power to drive the magnetic heat treatment device, and other related setting parameters.

In addition, in this embodiment, people having ordinary skill in the art may acquire sufficient teachings, suggestions, and other details related to the details of the device characteristics and details of the technology of the pre-surgical planning apparatus 100 according to content of the embodiments of FIG. 1 to FIG. 6B, and that detailed descriptions are not further provided hereinafter.

In view of the foregoing, in the pre-surgical planning apparatus and the pre-surgical planning method provided by the embodiments of the invention, estimation is performed through the estimation model according to the needle angle parameter, the needle characteristic parameter, and the thermal conductivity of the target object at least inputted by medical professionals, so as to generate the accurate ablation estimation result. Further, the pre-surgical planning apparatus may provide visualized ablation information through the display device, such that medical professionals may conveniently perform simulated parameter adjustment of the ablation estimation result according to the visualized ablation information. Therefore, medical professionals are expected to obtain the related setting parameters of the magnetic heat treatment apparatus through operating the pre-surgical planning apparatus provided by the embodiments of the invention.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A pre-surgical planning apparatus, adapted to estimate ablation information of a target object ablated by a magnetic heat treatment device, wherein the magnetic heat treatment device comprises a needle and a coil, and the pre-surgical planning apparatus comprises:
a storage device, configured to store an estimation model;
a processing device, coupled to the storage device, and configured to read the estimation model; and
an input device, coupled to the processing device and configured to provide a plurality parameters, wherein the parameters comprise a needle angle parameter, a needle characteristic parameter, and a thermal conductivity of the target object,
wherein the processing device inputs the parameters to the estimation model, so that the processing device executes the estimation model to estimate the ablation information of the target object ablated by the magnetic heat treatment device with a preset power and a preset length of time, and the ablation information comprises temperature curve information and temperature distribution information.

2. The pre-surgical planning apparatus as claimed in claim 1, wherein the estimation model is configured to simulate the needle induced by the coil, wherein the coil induces the needle according to the preset power, so that the coil heats the needle with a non-contact type method, and the target object and the needle are located inside a coil range of the coil.

3. The pre-surgical planning apparatus as claimed in claim 1, wherein the input device is further configured to provide a confirmation signal to the processing device, and the processing device determines whether to adjust the preset power according to the confirmation signal, so as to re-estimate the ablation information.

4. The pre-surgical planning apparatus as claimed in claim 3, wherein the processing device decreases the preset power if the processing device determines that an ablation result of the target object is excessive ablation according to the confirmation signal, and the processing device increases the preset power if the processing device determines that the ablation result of the target object is insufficient ablation according to the confirmation signal.

5. The pre-surgical planning apparatus as claimed in claim 1, wherein the processing device trains the estimation model in advance through a nonlinear regression, and the estimation model is a deep neutral network, wherein the estimation model has an input layer, a plurality of hidden layers, and an output layer, and the hidden layers comprise a plurality of influential parameters and a plurality of weights, wherein a number of the hidden layers is 3 to 60.

6. The pre-surgical planning apparatus as claimed in claim 1, further comprising:
a display device, coupled to the processing device and configured to display a temperature curve diagram and a temperature distribution diagram according to the temperature curve information and the temperature distribution information of the ablation information.

7. The pre-surgical planning apparatus as claimed in claim 1, wherein the needle characteristic parameter comprises a magnetic portion length and a needle diameter of the needle.

8. The pre-surgical planning apparatus as claimed in claim 1, wherein the parameters provided by the input device further comprise a location parameter, a target temperature, and a heating rate.

9. The pre-surgical planning apparatus as claimed in claim 1, wherein the thermal conductivity of the target object is determined by a body fat parameter of the target object.

10. A pre-surgical planning method, adapted to estimate ablation information of a target object ablated by a magnetic heat treatment device, wherein the magnetic heat treatment device comprises a needle and a coil, and the pre-surgical planning method comprises:
obtaining a plurality of parameters through an input device, wherein the parameters comprise a needle angle parameter, a needle characteristic parameter, and a thermal conductivity of the target object; and
inputting the parameters to an estimation model, and executing the estimation model to estimate the ablation information of the target object ablated by the magnetic heat treatment device with a preset power and a preset length of time, wherein the ablation information comprises temperature curve information and temperature distribution information.

11. The pre-surgical planning method as claimed in claim 10, wherein the estimation model is configured to simulate the needle inducted by the coil, wherein the coil induces the needle according to the preset power, so that the coil heats the needle with a non-contact type method, and the target object and the needle are located inside a coil range of the coil.

12. The pre-surgical planning method as claimed in claim 10, further comprising:
obtaining a confirmation signal through the input device and determining whether to adjust the preset power according to the confirmation signal so as to re-estimate the ablation information.

13. The pre-surgical planning method as claimed in claim 12, wherein the step of obtaining the confirmation signal through the input device and determining whether to adjust the preset power according to the confirmation signal so as to re-estimate the ablation information comprises:
decreasing the preset power if an ablation result of the target object is determined to be excessive ablation according to the confirmation signal; and
increasing the preset power if the ablation result of the target object is determined to be insufficient ablation according to the confirmation signal.

14. The pre-surgical planning method as claimed in claim 10, further comprising:
training the estimation model in advance through a nonlinear regression, and the estimation model being a deep neutral network,
wherein the estimation model has an input layer, a plurality of hidden layers, and an output layer, and the hidden layers comprise a plurality of influential parameters and a plurality of weights, wherein a number of the hidden layers is 3 to 60.

15. The pre-surgical planning method as claimed in claim 10, further comprising:
displaying a temperature curve diagram and a temperature distribution diagram according to the temperature curve information and the temperature distribution information of the ablation information through a display device.

16. The pre-surgical planning method as claimed in claim 10, wherein the needle characteristic parameter comprises a magnetic portion length and a needle diameter of the needle.

17. The pre-surgical planning method as claimed in claim 10, wherein the parameters provided by the input device further comprise a location parameter, a target temperature, and a heating rate.

18. The pre-surgical planning method as claimed in claim 10, wherein the thermal conductivity of the target object is determined by a body fat parameter of the target object.
